(19) United States
(12) Patent Application Publication
(10) Pub. No.: US 2003/0185814 A1
(43) Pub. Date: Oct. 2, 2003

(54) Ceramidase

(76) Inventors: Kay Hofmann, Koln (DE); Marcus Conradt, Pretoria (ZA)

Correspondence Address: JACOBSON HOLMAN PLLC, 400 SEVENTH STREET N.W., SUITE 600, WASHINGTON, DC, 20004, US

(21) Appl. No.: 10/182,447

(22) PCT Filed: Jan. 27, 2001
(86) PCT No.: PCT/EP01/00900

(30) Foreign Application Priority Data
Jan. 27, 2000 (DE) 10003293.1
Mar. 9, 2000 (DE) 10011392.3

(51) Int. Cl. A01K 67/00; C07H 21/04; A61K 38/47; C12N 9/24; C12N 5/06

(52) U.S. Cl. 424/94.61; 435/200; 536/23.2; 435/354; 800/8

(57) ABSTRACT

A ceramidase having the following structural motif

Z¹-X1-H-X2-T-L-X2-X2-X2-X2-Q-X2-X2-D-E,-Z²

wherein the X1 position is an aromatic amino acid (F, Y or W), all X2 positions may be any independent amino acids, and Z¹ and Z² may be any polypeptides.

The present invention relates to a ceramidase.

Ceramidase cleaves ceramide and produces sphingosine, which is subsequently further converted into sphingosine-1-phosphate. Various ceramidase activities have been described:

1. an acid lysosomal ceramidase which further degrades the ceramide formed by acid sphingomyelinase;

2. a non-acid membrane-bound ceramidase activity;

3. secreted neutral and alkaline ceramidase activities.

WO 00/56891 relates to speculative human transmembrane proteins. It discloses a sequence which corresponds to a ceramidase, but without assigning functionality to it. Koch, Jürgen et al. in Journal of Biological Chemistry (271) 33110-33115, Rosenberg A. in Proceedings of the Federation of European Biochemical Societies 1980, L. Babab Sama et al. in Journal of Biological Chemistry (274), pages 27948-27955, Jada Y. et al. in Journal of Biological Chemistry (270), pages 12677-12684, also report on ceramidases and their characterization.

According to the invention, a ceramidase is provided having the following structural motif

Z¹-X1-H-X2-T-L-X2-X2-X2-X2-Q-X2-X2-D-E,-Z²

wherein the X1 position is an aromatic amino acid (F, Y or W), all X2 positions may be any independent amino acids, and Z¹ and Z² may be any polypeptides.

The proteins claimed according to the invention contain the motif mentioned in claim 1. These include proteins which have been modified in the side chains, especially with phosphorylations, glycosylations, amidizations and other derivatizations in the side chains. In addition, the skilled person will recognize that fragments which also ensure the biological functions of the proteins according to the invention can be prepared by modifications in the primary structure. These include, for example, fragmentations in the region around the motif claimed according to the invention. The corresponding biologically active fragments can be readily established by the skilled person by appropriate experiments. These include, for example, site-directed mutagenesis by means of which structural variants which also have biological activity can be prepared. In addition, in terms of protein chemistry, exchanges of amino acids or sequences or motifs within the primary structure also suggest themselves. Such exchanges are well-known to the skilled person and comprise, for example, conservative exchanges in which, for example, serine is exchanged against threonine. Of course, there are further groups known to the skilled person which are candidates for exchange. For example, the members of the groups of non-polar amino acids, polar amino acids, aromatic amino acids are respectively interchangeable without any substantial changes in biological function to be expected.

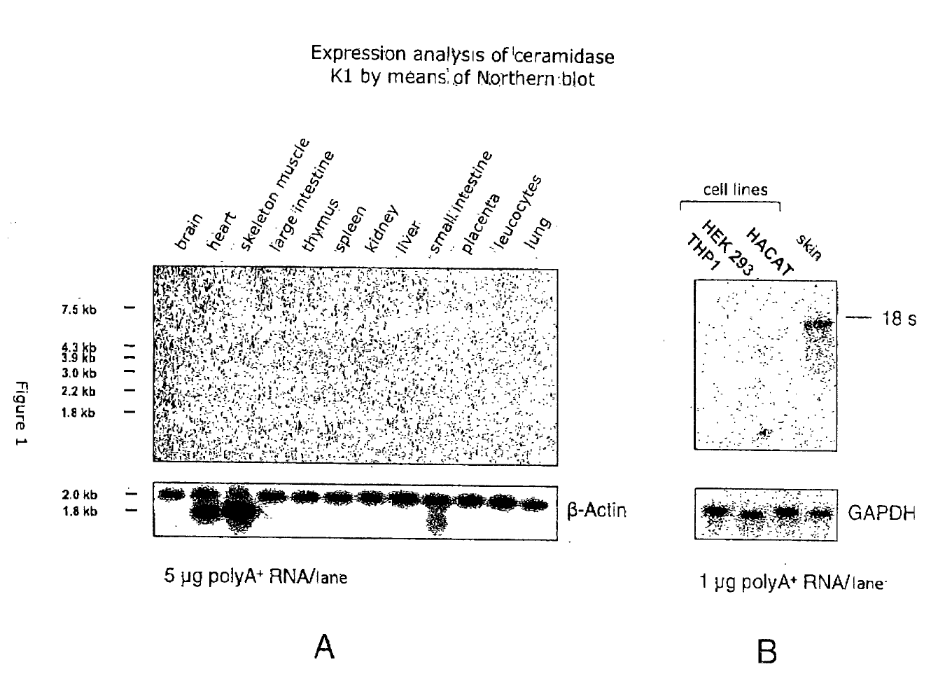

FIG. 1 shows the result of Northern blot analysis. Part A (left) shows that no signal from ceramidase can be seen in a wide variety of human tissues. Part B (right) shows that a strong signal can be seen in skin RNA, which signal is absent in the human cell lines shown.

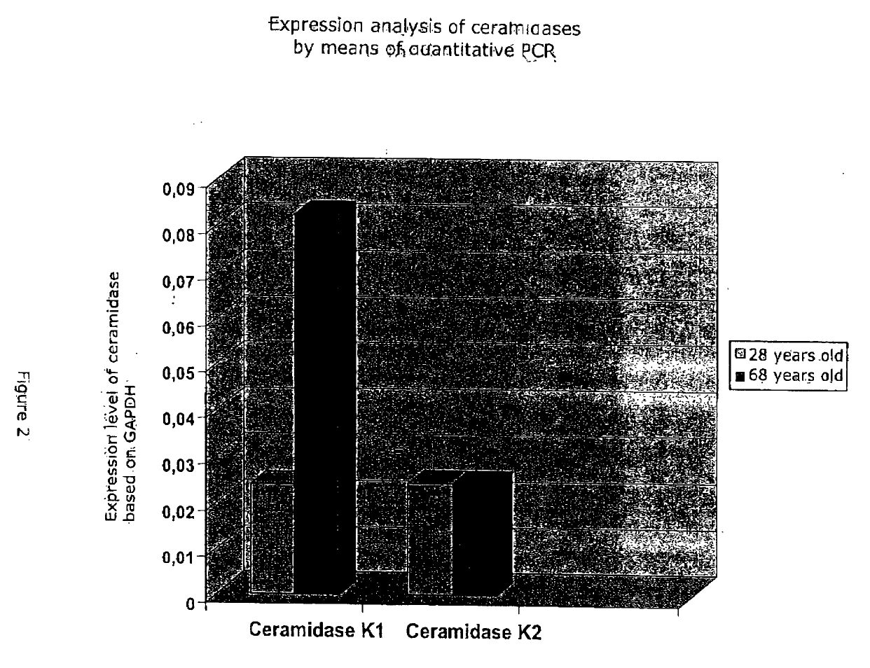

FIG. 2 shows the result of a quantitative PCR analysis of the skin-specific ceramidase K1 and in comparison to ceramidase K2. The expression level of ceramidase K1 in the skin is age-dependent; in the skin of a 68-year-old human, a stronger signal can be observed than in the skin of a 28-year-old human. Ceramidase K2 does not exhibit any age dependence.

Preferred are transmembrane ceramidases.

Preferred ceramidases have the following sequence:

| | |
|---|---|
| Ceramidase K1 (human protein) | SEQ ID NO: 1; |
| Ceramidase K2 (human protein) | SEQ ID NO: 2; |
| Ceramidase K4 (human protein) | SEQ ID NO: 3; |

The invention also relates to nucleic acids coding for ceramidase, especially those having the sequences:

| | |
|---|---|
| Ceramidase K1 | SEQ ID NO: 4; |
| Ceramidase K2 | SEQ ID NO: 5; |
| Ceramidase K4 | SEQ ID NO: 6. |

The invention also relates to a nucleic acid which is complementary to these nucleic acids.

A further aspect of the invention is a medicament, cosmetic or diagnostic agent containing the ceramidase according to the invention or a nucleic acid according to the invention, excluding the ceramidase SEQ ID NO: 2, i.e., SEQ ID NO: 5.

The medicaments, cosmetic and diagnostic agents are suitable for the diagnostics of diseases, for the treatment or prevention of diseases associated with a ceramidase defect, especially in connection with diseases accompanied by a defective cell proliferation (e.g., cancer), diseases accompanied by a defect in the ceramide layer of the skin, such as neurodermitis, eczema, psoriasis, and for the well-aimed affection of the permeability barrier by ceramidase or ceramidase activators, e.g., for the transcutaneous administration of substances.

The gene for ceramidase K4 resides on chromosome 2 in the region 2q33q34. This region also contains the gene for the skin diseases “lamellar ichthyosis ICR2B”.

The ceramidase K4 according to the invention is particularly suitable for the diagnostics and therapy of this latter disease, with which it is presumably connected in a causal relationship.

The invention also relates to a cell line which overexpresses the ceramidases according to the invention, and to a transgenic animal with overexpression or gene deficiency or gene defect for the ceramidases according to the invention.

### EXAMPLES

Cloning of the Whole cDNA:

The cDNA of Cdase1 was amplified by means of PCR. 5 μg of human skin RNA, which had been previously subjected to reverse transcription, served as the template. The following primers were used for amplification:

| | | |
|---|---|---|
| cer1 atg2 s: | caagATGCCTAGCATCTTCGCC | Seq. ID No 7 |
| cert e2 as: | caggtctcagcagtccttgtcatc | Seq. ID No 14 |

The amplified cDNA was subcloned, and the sequence was subsequently verified.

Tissue Distribution

The tissue distribution of Cdase1 was analyzed by the Northern blot technique. Thus, there was used, on the one hand, a commercially available blot (Clontech Laboratories Inc., Catalog #: 7780-1), and on the other hand, a blot prepared from RNA from cell lines and human skin (1 μg each of poly-A⁺ RNA). The first 668 bp of the cDNA was used as a probe. The cDNA fragment was labeled radioactively. Hybridization took place over night at 65° C. The radioactive signals were measured by means of the Cyclone Storage Phosphor System (both supplied by Packard Instrument Company, Dreieich, Germany) and evaluated with the program OptiQuant. The blots were normalized with either β-actin or GAPDH.

Cell Lines Which Stably Overexpress Cdase1:

The whole cDNA was cloned into a eukaryotic expression vector under the control of CMV promoter. HEK293 cells were electroporated with the construct and subsequently selected with G418. Cell clones which survived the selection were examined for Cdase1 overexpression.

Age-Dependent Expression of Cdase1:

The expression levels in young and old skin were established by means of real time PCR using Taq-man (Perkin Elmer). GAPDH served as an internal standard.

The Following Primers Were Employed:

| | | | |
|---|---|---|---|
| CER1.SEQ-456F: | GCA TTG CCC TGC ACA TTC T | | Seq ID No 8 |
| CER1.SEQ-526R: | GTG CCG AAG CTC CTT ATT GC | | Seq. ID No 9 |
| tmcer1: | CATCGTGTGCCAGGABTACAGGAAGACC | [5′]6_FAM [3′]TAMRA | Seq. ID No 10 |
| GAPDHS: | GAAGGTGAAGGTCGGAGT | | Seq. ID No 11 |
| GAPDHR: | GAAGATGGTGATGGGATTTC | | Seq. ID No 12 |
| GAPDHT: | CAAGCTTCCCGTTCTCAGCC | [5′]6_FAM [3′]TAMRA | Seq. ID No 13 |

1. A ceramidase having the following structural motif
Z¹-X1-H-X2-T-L-X2-X2-X2-X2-Q-X2-X2-D-E,-Z²
wherein the X1 position is an aromatic amino acid (F, Y or W), all X2 positions may be any independent amino acids, and Z¹ and Z² may be any polypeptides.

2. The ceramidase according to claim 1, characterized by comprising the sequences SEQ ID NOS: 1, 2 and/or 3.

3. A nucleic acid coding for ceramidase according to either of claims 1 or 2, especially a nucleic acid having the sequence of SEQ ID NOS: 4, 5 and/or 6.

4. A nucleic acid, characterized by being complementary to the nucleic acid according to claim 3.

5. A medicament, cosmetic or diagnostic agent containing the ceramidase according to claim 1 or 2 or the nucleic acid according to claim 3 or 4.

6. Use of the medicament or diagnostic agent according to claim 5 for the diagnostics of diseases associated with a ceramidase defect, for the treatment or prevention of diseases associated with a ceramidase defect, especially in connection with diseases accompanied by a defective cell proliferation (e.g., cancer), diseases accompanied by a defect in the ceramide layer of the skin, such as neurodermitis, eczema, psoriasis, and for the well-aimed affection of the permeability barrier by ceramidase or ceramidase activators, e.g., for the transcutaneous administration of substances.

7. Use of the medicament or diagnostic agent according to claim 5 for the diagnostics of ichthyosis, especially lamellar ichthyosis ICR2B.

8. Use of the ceramidase according to claim 1 or 2 as a cosmetic agent.

9. A transgenic animal exhibiting overexpression (gain of function) or gene deficiency or gene defect (loss of function) for a ceramidase according to either of claims 1 or 2.

10. A cell line, characterized by overexpressing a ceramidase according to either of claims 1 or 2.


SEQUENCE LISTING (14 sequences)

SEQ ID NO: 1 - protein, 264 aa, Homo sapiens
MPSIFAYQSSEVDWCESNFQYSELVAEFYNTFSNIPFFIFGPLMMLLMHPYAQKRSRYIYVVWVLFMIIGLFSMYFHMTLSFLGQLLDEIAILWLLGSGYSIWMPRCYFPSFLGGNRSQFIRLVFITTVVSTLLSFLRPTVNAYALNSIALHILYIVCQEYRKTSNKELRHLIEVSVVLWAVALTSWISDRLLCSFWQRIHFFYLHSIWHVLISITFPYGMVTMALVDANYEMPGETLKVRYWPRDSWPVGLPYVEIRGDDKDC

SEQ ID NO: 2 - protein, 267 aa, Homo sapiens
MAPAADREGYWGPTTSTLDWCEENYSVTWYIAEFWNTVSNLIMIIPPMFGAIQSVRDGLEKRYIASYLALTVVGMGSWCFHMTLKYEMQLLDELPMIYSCCIFVYCMFECFKIKNSVNYHLLFTLVLFSLIVTTVYLKVKEPIFHQVMYGMLVFTLVLRSIYIVTWVYPWLRGLGYTSLGIFLLGFLFWNIDNIFCESLRNFRKKVPPIIGITTQFHAWWHILTGLGSYLHILFSLYTRTLYLRYRPKVKFLFGIWPVILFEPLRKH

SEQ ID NO: 3 - protein, 273 aa, Homo sapiens
MGAPHWWDQLQAGSSEVDWREDNYTIVPAVAEFYNMISNVLFFILPPICMCLFRQYATCFNSGIYLIWLLVVAGIGSVYFHATLSFLGQMLDELAVLWVLMCASVMWFPRRYLPKIFRNDQGRFKVVCVLSAVMTCLAFVKPAINNISLMTLGVPCAALLITELKRCDNMRVFKLGLFSGLWWTLALFCWISDRAFCELLSSFNFPYLHCMWHILICLAAYLGCVCFAYFDAASEIPEQGPVIKFWPSEKWAFIGVPYVSLLCANKKSSVKTT

SEQ ID NO: 4 - DNA, 792 bp, Homo sapiens
atgcctagcatcttcgcctatcagagctccgaggtggactggtgtgagagcaacttccagtactcggagctggtggccgagttctacaacacgttctccaatatccccttcttcatcttcgggccactgatgatgctcctgatgcacccgtatgcccagaagcgctcccgctacatttacgttgtctgggtcctcttcatgatcataggcctgttctccatgtatttccacatgacgctcagcttcctgggccagctgctggacgagatcgccatcctgtggctcctgggcagtggctatagcatatggatgccccgctgctatttcccctccttccttggggggaacaggtcccagttcatccgcctggtcttcatcaccactgtggtcagcacccttctgtccttcctgcggcccacggtcaacgcctacgccctcaacagcattgccctgcacattctctacatcgtgtgccaggagtacaggaagaccagcaataaggagcttcggcacctgattgaggtctccgtggttttatgggctgttgctctgaccagctggatcagtgaccgtctgctttgcagcttctggcagaggattcatttcttctatctgcacagcatctggcatgtgctcatcagcatcaccttcccttatggcatggtcaccatggccttggtggatgccaactatgagatgccaggtgaaaccctcaaagtccgctactggcctcgggacagttggcccgtggggctgccctacgtggaaatccggggtgatgacaaggactgc

SEQ ID NO: 5 - DNA, 801 bp, Homo sapiens
atggctccggccgcggaccgagagggctactggggccccacgacctccacgctggactggtgcgaggagaactactccgtgacctggtacatcgccgagttctggaatacagtgagtaacctgatcatgattatacctccaatgttcggtgcaattcagagtgttagagacggtctggaaaagcggtacattgcttcttatttagcactcacagtggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccaatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcat

SEQ ID NO: 6 - DNA, 822 bp, Homo sapiens
atgggcgccccgcactggtgggaccagctgcaggctggcagctcggaggtggactggcgcgaggacaactacaccatcgtgcctgctgtcgccgagttctataacatgatcagcaatgtcttatttttcattttaccgcccatctgcatgtgcttgtttcgtcagtatgcaacatgcttcaacagcggcatctacttaatctggctcttggttgtagcgggaattggatccgtctacttccatgcaacccttagtttcctgggtcagatgcttgatgaacttgcagtcctttgggttctgatgtgtgcttcggtcatgtggttccccagaaggtatctaccaaagatctttcggaatgaccagggtaggttcaaggtggtggtctgtgtcctgtctgcagttatgacgtgcctggcatttgtcaagcctgccatcaacaacatctctctgatgaccctgggagttccttgcgctgcactgctcatcacagagctaaagaggtgtgacaacatgcgtgtgtttaagctgggcctcttctcgggcctctggtggaccctggccctgttctgctggatcagtgaccgagctttctgcgagctgctgtcatccttcaacttcccctacctgcactgcatgtggcacatcctcatctgccttgctgcctacctgggctgtgtatgctttgcctactttgatgctgcctcagagattcctgagcaaggccctgtcatcaaattctggcccagcgagaaatgggccttcattggtgtcccctatgtgtccctcctgtgtgccaacaagaaatcatcagtcaagaccacg

SEQ ID NO: 7 - DNA, 22 bp, Artificial Sequence
  Primer
caagatgcctagcatcttcgcc

SEQ ID NO: 8 - DNA, 19 bp, Artificial Sequence
  Primer
gcattgccctgcacattct

SEQ ID NO: 9 - DNA, 20 bp, Artificial Sequence
  Primer
gtgccgaagctccttattgc

SEQ ID NO: 10 - DNA, 28 bp, Artificial Sequence
  Primer
catcgtgtgccaggabtacaggaagacc

SEQ ID NO: 11 - DNA, 18 bp, Artificial Sequence
  Primer
gaaggtgaaggtcggagt

SEQ ID NO: 12 - DNA, 20 bp, Artificial Sequence
  Primer
gaagatggtgatgggatttc

SEQ ID NO: 13 - DNA, 20 bp, Artificial Sequence
  Primer
caagcttcccgttctcagcc

SEQ ID NO: 14 - DNA, 24 bp, Artificial Sequence
  Primer
caggtctcagcagtccttgtcatc